(19) United States
(12) Patent Application Publication
(10) Pub. No.: US 2022/0160948 A1
(43) Pub. Date: May 26, 2022

(54) TREATMENT APPARATUS AND METHOD FOR EXTRACORPOREAL IMMUNOTOLERANCE ENHANCING BLOOD TREATMENT

(71) Applicant: Fraunhofer-Gesellschaft zur Forderung der angewandten Forschung e.V., Muenchen (DE)

(72) Inventors: Heiko ZIMMERMANN, Sulzbach (DE); Julia NEUBAUER, Wuerzburg (DE); Benjamin FISCHER, Wuerzburg (DE)

(21) Appl. No.: 17/439,675

(22) PCT Filed: Mar. 13, 2020
(86) PCT No.: PCT/EP2020/056855

§ 371 Date: Sep. 15, 2021

(30) Foreign Application Priority Data
Mar. 15, 2019 (DE) 10 2019 106 651.4

(51) Int. Cl. A61M 1/36

(52) U.S. Cl. CPC A61M 1/3689; A61M 1/3621; A61M 2202/09; A61M 2202/0437; A61M 1/3672

(57) ABSTRACT

The invention relates to a treatment device (100) which is designed for extracorporeal blood treatment of blood of a subject (2) for an immunotolerance enhancement of the subject (2), comprising at least one container (10) which has an interior (11) for receiving a blood sample (1) of blood of the subject (2) and an exchange device (20) designed to supply and/or discharge the blood sample (1) into or out of the interior (11) of the container (10) and comprising a cell exposure device (30) which is positioned in the container (10) and has biological cell material (31) with surface molecules, said cell material being arranged in the interior (11) of the container (10) for a material interaction with the blood sample (1), including a reaction of endogenous immune cells in the blood with the surface molecules of the cell material. The invention also relates to a kit for extracorporeal blood treatment and to a method for operating the treatment device.

The invention relates to a treatment apparatus which is designed for extracorporeal blood treatment of blood from a subject for an immunotolerance enhancement of the subject, to a kit for extracorporeal blood treatment and to a method for immunotolerance enhancement by extracorporeal blood treatment. The invention has applications in medicine and biochemistry, in particular in enhancing the immunotolerance of organisms to exogenous or endogenous implants or in autoimmune disorders.

In the present description, reference is made to the following prior art which illustrates the technical background of the invention.
[1] M. Zouali “Immunological Tolerance: Mechanisms”, in: Encyclopedia of Life

Sciences. John Wiley & Sons, Ltd, Chichester. 2001;
[2] E. Shaban et al. in “Kidney Dis.” 4, 205-213, 2018;
[3] DE 10 2017 210 134 A1; and
[4] U.S. Pat. No. 4,685,900.

The occurrence of an immune reaction (immune response) as a response of an organism's immune system to exogenous or even endogenous substances (antigens) is known in general. The absence or reduction of an immune response to antigens is referred to as immunotolerance (see e.g. [1]). When an immune response causes damage to the organism, immunosuppressive treatment (immunosuppression) is usually undertaken using drugs and/or irradiation. This is intended for example to prevent rejection reactions after allogeneic transplants, in particular organ transplants. However, because of side effects, immunosuppressive treatments have considerable disadvantages for the organism being treated, e.g., the increased risk of infections which are difficult to control, an increased risk of suffering from cancer, and a general worsening of quality of life.

There is therefore interest in avoiding or reducing immunosuppressive treatments. To this end, to date, prior to an allogeneic transplant, donor material is sought which is adapted to the recipient's immune system due to the antigens contained in said donor material, and which means that a lesser, or no, immune response is expected. However, a search of this kind is only of limited success in the attempt to minimize immunosuppression, because of the small number of available donor organs or in the occurrence of autoimmune disorders.

The use of immunotherapies in cancer is also known. In extracorporeal immunomodulation, blood is taken from the diseased organism and modified outside the body during a treatment. During the treatment, T cells in the blood are modified such that they have an increased ability to attack cancer cells. After returning the treated blood to the organism, the immune system's sensitivity to cancer cells is increased, and therefore the body's own defenses against cancer are reinforced. Immunomodulation has proven promising in cancer treatment by intensifying immune responses. However, immunomodulation is unsuitable for suppressing immune responses or for increasing immunotolerance, e.g., in transplants.

It is known from transplant medicine that immune responses decrease over time following an allogeneic transplant, such that immunosuppressive treatment can be ended if the progression is favorable. Furthermore, it is known that a high antigen concentration acts favorably on the development of increased immunotolerance [1] and that active substances, e.g., the active substance rapamycin [2], can promote the development of immunotolerance.

It is also known to carry out specific immune therapies for treating allergies (hypersensitization), where exogenous allergens are supplied to the diseased organism in order to increase immunotolerance. However, such immunotherapies have the disadvantage of having high risk of side effects, in particular due to the introduction of allergens into the organism.

A system for extracorporeal blood treatment with three blood treatment apparatuses, which comprise an adsorber device and/or a plasma separator, a dialysis device and a gas exchange device, is known from [3]. The adsorber device can be designed for the adsorption e.g., of antibodies. The application of this system, similar to other technologies for hemodialysis, is limited to modifying blood e.g., in metabolic disorders. [4] also describes a system for extracorporeal blood treatment in which biopolymers which have been provided with biologically active substances remove disease components from the blood. An enhancement of immunotolerance is not possible with the systems according to [3] or [4].

The objective of the invention is to provide an improved treatment apparatus for extracorporeal blood treatment, an improved kit for extracorporeal blood treatment and/or an improved method for extracorporeal blood treatment, by means of which apparatus, kit and method the disadvantages of conventional techniques are avoided and which in particular enable an enhancement of the immunotolerance of a subject, a decrease in or the exclusion of side effects in the blood treatment, a reduced risk of rejection reactions and/or a gentle treatment of autoimmune diseases.

These objectives are each accordingly achieved by a treatment apparatus for extracorporeal blood treatment, a kit for extracorporeal blood treatment and a method for extracorporeal blood treatment which have the features of the independent claims. Advantageous embodiments and applications of the invention result from the dependent claims.

According to a first general aspect of the invention, the above objective is achieved by a treatment apparatus which is designed for extracorporeal blood treatment of blood from a subject for an immunotolerance enhancement of the subject. Extracorporeal blood treatment is a change in a blood sample of the blood from the subject in the treatment apparatus outside the body (organism) of the subject. The subject can be a human organism (in particular a patient, transplant recipient) or an animal organism (mammalian organism), e.g., for evaluating a cell therapy with animal testing. The treatment apparatus comprises at least one container (also referred to as cartridge) having rigid and/or flexible container walls, wherein the container has an interior (also referred to as reaction chamber) for receiving a blood sample from the subject. The interior has a volume e.g., in the range of 1 ml to 1 l. The container is generally a vessel with can be closed to the outside, in particular closed in a sterile manner, in which the blood sample is arranged in stationary or moved manner during the extracorporeal treatment. The container is further provided with an exchange device which is designed to supply and/or discharge the blood sample into or out of the interior of the container for blood exchange with the organism. The blood exchange with the organism can be a direct exchange in which the treatment apparatus is directly connected to the subject's circulation, or an indirect exchange in which the treatment apparatus is connected to a separate supply and/or collection reservoir.

According to the invention, the treatment apparatus has a cell exposure device. The cell exposure device has biological cell material which is arranged in the container in an exposed manner towards the interior thereof in such a way that the blood sample can materially interact with the biological cell material in the interior. The cell exposure device is arranged in the container, i.e., positioned within the container walls or integrated into one of the container walls. The biological cell material is arranged with the cell exposure device in a locally restricted manner in a cell material region, which is at least locally spatially delimited from the interior. The cell exposure device is configured such that the blood comes into contact with the cell material or surface molecules, e.g., antigens, thereof, in particular blood cells touch surfaces of the cell material or surface molecules of the cell material, without cells of the cell material entering the interior. The material interaction between the blood and the cell material takes place by mutual contacting at the interface between the interior and the cell material region or by the ingress of surface molecules of the cell material into the interior.

The cell exposure device provides exposure to the cell material to which the blood sample is exposed in the interior. Advantageously, blood sample-cell material interactions take place, in particular a reaction of the endogenous immune cells in the blood sample with the surface antigens of the presented cell material, which causes immunomodulation but which does not act directly on the subject due to the extracorporeal treatment. The blood is adapted to the cell material (in particular antigens of the cell material) outside the body (adaptive immune response), and as a result acquires enhanced immunotolerance. By returning the treated blood to the subject, the enhanced immunotolerance is transmitted to the subject. The immunotolerance of the subject is established directly by the treated blood sample and/or by an interaction of the treated blood sample, in particular of T cells activated by the treatment, with the subject's immune system (e.g., in the lymph nodes). Single or multiple treatment of the subject's blood increases the immunotolerance thereof, such that transplants can be carried out with a lower or absent risk of rejection reactions, or autoimmune disorders can be alleviated or treated.

The treatment apparatus is preferably a disposable item which can be used once or more exclusively for a single subject and disposed of after use.

According to a second general aspect of the invention, the above object is achieved by a kit for extracorporeal blood treatment, comprising at least one treatment apparatus according to the first general aspect of the invention, wherein the exchange device is configured for direct use at a treatment location, e.g., in a doctor's surgery or clinic. To this end, the exchange device preferably has at least one tube and at least one sterile injection needle. The at least one treatment apparatus or at least the cell exposure device of the at least one treatment apparatus can be provided in the kit for extracorporeal blood treatment in the frozen state. If the treatment apparatus, in particular the cell exposure device with the biological cell material, is provided in the frozen state, the treatment apparatus is thawed before the blood sample is introduced into the container.

According to a third general aspect of the invention, the above object is achieved by a method for operating the treatment apparatus according to the first general aspect of the invention. A blood sample to be treated of blood from a subject is provided, and added to the container of the treatment apparatus. The blood sample in the container is treated by an interaction of the blood sample, in particular of the immune cells of the blood sample, with the biological cell material. The duration of the treatment is preferably selected in the range from 5 minutes to 8 hours. After the treatment, the treated blood sample is characterized by an enhanced immunotolerance, i.e., a reduced immune response to the cell material. The blood sample is subsequently removed from the container.

The term “blood sample” relates to a predetermined volume of blood from the subject, as is provided by removal from the blood circulation and optionally intermediate storage in a reservoir, or to a suspension of cellular components, in particular immune cells, from the subject's blood. In the latter case, the suspension can be obtained e.g., after the removal of blood from the circulation by separating the blood and removing components which do not have an effect on the immune response, e.g., blood plasma or other components. For enhancing immunotolerance, a blood sample with an overall volume in the range from 1 ml to 1 l is preferably treated.

The invention affords the following substantial advantages. For the first time, immunotolerance is induced via the blood exclusively using self-regulation of the subject, in particular of a transplant recipient. In contrast to usual immunotherapies, e.g., hypersensitization for allergies, according to the invention the blood-cell material interaction takes place outside the body in the treatment apparatus, in particular in the container. Further, in contrast to usual therapeutic approaches for promoting immunotolerance, no viral vectors are (directly or indirectly) introduced into the subject. No exogenous material, or only extremely small amounts thereof, is/are transmitted back into the subject's body, and therefore severe side effects, e.g., anaphylactic shock, are ruled out.

Advantageously, targeted reduction in the immune response and in the mediation of immunotolerance in a transplant recipient (subject) takes place. Here, an immune response to biological, e.g., exogenous cell material (e.g., cells, cell components, cell mixtures, tissue or whole organs) is reduced by single or repeated exposure of one or more blood samples from the subject to the e.g., exogenous cell material. Since immune responses to exogenous material often cause drastic immune responses in the recipient, the parts of the immune system found in the blood are exposed to the exogenous biological material in the treatment apparatus, and not in the subject.

The method for reducing the immune response of the subject to the cell material preferably comprises the following steps. Firstly, blood is taken repeatedly from the subject, preferably before a transplant of exogenous cell material, and this blood is transferred into the treatment apparatus. In the treatment apparatus, components of the exogenous biological material to be transplanted, or the exogenous biological material itself, which originates from the material/tissue/organ/donor to be transplanted or emulates same, are located adjacent to the interior. For example, this material could be produced from stem cells (e.g., induced pluripotent stem cells, mesenchymal stem cells) or from cells derived from stem cells. Thus, the recipient can be immunologically habituated to the transplant even before the actual transplant.

While the blood sample is incubated in the interior of the container, the immunocompetent components of the blood react to the presented antigens of the cell material. The amount of exogenous cell material can be adapted here to the application or to the subject's immune response. Thus, locally high antigen concentrations can be reached in the container, which are considered to be particularly conducive to promoting the development of immunotolerance (see [1]). However, low antigen concentrations can also be produced by less exogenous cell material being contained in the container. In the context of a therapy for developing immunotolerance, it is also possible to use plural containers with different amounts of exogenous cell material.

After incubation, the blood sample, optionally after processing, is transferred back into the subject, and with this process step the immunocompetent components of the blood sample are also transferred back into the subject. The memory function and the regulatory effect of the immunocompetent components (T and B cells, regulatory T cells, memory T cells, macrophages, natural killer cells and dendritic cells) are thus transferred back into the body and can interact there with the subject's immune system and/or influence the immunotolerance thereof.

Repeated exposure and returning to the patient thus induce an immunomodulatory effect, which advantageously leads to immunotolerance even before the transplantation. It is particularly advantageous that the immune response takes place outside the subject's body, but can be carried out with biological cell material from the donor.

According to a preferred embodiment of the invention, if the cell exposure device has a separating layer by means of which the biological cell material is separated from the interior, this may afford advantages in terms of preventing the introduction of the cell material or larger components thereof, such as cell organelles, membrane fragments or the like, into the blood sample. By means of the separating layer, which particularly preferably comprises a semi-permeable membrane or a semi-permeable grid, the biological cell material is held on the cell exposure device, wherein the separating layer is adapted for a substance exchange between the biological cell material and the blood sample.

According to a preferred variant of the invention (hereinafter: first embodiment), the cell exposure device has a support (substrate) on which the biological cell material is arranged in an adherent state. Advantageously, the cell material bound to the support is easy to handle when preparing the cell exposure device, e.g., in a separate incubator, and/or when arranging in the container. A further advantage lies in the inherent attachment of the cell material, such that the cell material region which is spatially delimited from the interior is formed by the surface of the cell material on the support or the surface of a separating layer on the cell material bound to the support. The support with the cell material can be directly arranged in the interior of the container. The material, e.g., hydrogel, plastic, glass or ceramic, the structure, e.g., elastic, surface-structured, compact, porous and/or fibrous, and the form of the support can be selected from a plurality of variants. For example, a flexible support can be configured for a deformation in the interior and thus for movement of the blood sample relative to the cell material, and/or for surface area enlargement, e.g., by folding, in order to present more biological cell material. Furthermore, a support can comprise a plurality of partial substrates, each of which carries the cell material and which are arranged mutually spaced apart, such that a large internal surface area of the cell material can be provided in the interior of the container. Furthermore, a support can comprise a porous material with an inner surface, which carries the cell material, and with a porosity which permits the free flowing of the blood sample in the interior through the porous material. In this case, the optionally provided separating layer may be arranged exclusively on the outer surface of the porous material.

Advantageously, the support can be provided with a binding layer which promotes adherent binding of induced pluripotent stem cells (iPS cells), adult stem cells and/or cells differentiated therefrom. The binding layer affords the advantage of improved attachment of the cell material to the support. In particular when providing the binding layer, it is possible to dispense with the optionally provided separating layer of the cell exposure device.

According to an alternative variant of the invention (hereinafter: second embodiment), the cell exposure device, alternatively or in addition to the support, has a suspension space separated from the interior, in which suspension chamber the biological cell material is arranged in a suspended state and/or on carrier beads (carrier pearls, microcarriers). The separating layer of the cell exposure device is preferably provided between the interior and the suspension space. The second embodiment affords the advantage of moving the biological cell material with simultaneous separation of cell material and the blood sample in the interior of the container.

A further essential advantage of the invention is that plural biological cell materials are available for treating the subject's blood. The biological cell material may be of human origin, from an exogenous donor, or from the subject themself. Alternatively or in addition, the biological cell material may be of animal origin, e.g., in order to increase the subject's immunotolerance for a xenotransplantation (transplantation of animal material into humans, or vice-versa) or in order to treat blood from an animal subject, in particular for research purposes. The biological cell material can comprise intact cells and/or cell components, e.g., cell membranes. According to further alternatives which can be provided separately or in combination, the biological cell material can comprise exogenous biological cell material, endogenous biological cell material from the subject, cell-equivalent iPS cells and/or iPS cell components, organ-equivalent differentiated iPS cells and/or iPS cell components, organ-equivalent differentiated adult stem cells or cell components thereof, biological cells and/or cell components, which trigger antigen-antibody responses in the blood, exogenous cells or organ parts, e.g. tissues, as are provided for allogeneic transplantation into the subject, and/or components thereof, a composition of cell material which contains various cell types which carry characteristic HL antigens from a predetermined reference group of subjects, in particular a population group, and cells which have been genetically modified to undergo cell death which can be stimulated externally and/or in a time-dependent manner, e.g. induced by light or active substances. Alternatively or in addition, the cell material can comprise active substances which have an immunotolerance-enhancing effect or have a supporting function in inducing immunotolerance. For example, a strengthening of the response of the blood, an extension of the residence time of specific blood components, an enrichment of specific blood components, an extension of immunotolerance, an inhibition of blood coagulation, an intensification of immunotolerance and/or a mediation of immunotolerance after returning the modulated blood (downstream strengthening) can be provided. The cell material can comprise in particular proteins, biologically active substances, e.g. rapamycin, RNA, DNA and/or viruses.

It is particularly advantageous to use cell-equivalent iPS cells and/or iPS cell components (i.e. iPS cells or iPS cell components which carry the same antigens as the cells from which the iPS cells were obtained), organ-equivalent differentiated iPS cells and/or cell components (i.e. differentiated iPS cells or iPS cell components which carry the same antigens as the organs from which the iPS cells were obtained) and/or organ-equivalent differentiated adult stem cells or cell components (i.e. differentiated adult stem cells or corresponding cell components which carry the same antigens as the cells from which the adult stem cells were obtained), since these carry the same antigens as the corresponding cells or organs of the donor organism, and are therefore suitable for the desired interaction with the blood sample from the subject in a delimited space, such as the interior of the container.

According to the invention, if the cell material contains a composition of different cell types which carry characteristic HL antigens, e.g. of a population group, an enhancement of immunotolerance can be achieved which has a higher probability of being effective for freely selectable transplants from donors of this population group. Advantageously, this makes it possible to carry out the treatment independently of the presence of the actually anticipated donor material. Even if the antigens of a transplant are not precisely found with the composition of different cell types, an effective enhancement of immunotolerance is possible.

The use of cells which have been genetically modified to undergo cell death which can be stimulated externally and/or in a time-dependent manner, e.g. induced by light or active substances, has the advantage that any cell material that may inadvertently enter the subject can be rendered ineffective by light or active substances or the passage of time.

The treatment apparatus can further be designed to force a reduction in the immune response in autoimmune disorders. In this case, the affected tissue from the patient is used directly or produced from induced pluripotent or adult stem cells and conveyed into the interior of the container. Subsequently, blood is taken at least once, preferably several times, in each case followed by treatment in the treatment apparatus and return to the body.

A further particular advantage of the invention is that the amount and/or concentration of the biological cell material in the treatment apparatus can be adjusted depending on a given or achieved immunotolerance of the subject. The cell exposure device is loaded with the biological cell material such that the enhancement of the subject's immunotolerance is optimized.

According to a further advantageous embodiment of the invention, the exchange device has an inlet line and an outlet line which are in fluidic connection with the interior and are arranged mutually spaced apart. This enables the container to be advantageously configured as a through-flow system. Particularly preferably, the inlet and outlet lines are arranged on mutually opposite sides of the container. Particularly preferably, the inlet line and the outlet line each have a shut-off element, e.g. a valve. Alternatively or in addition, the exchange device can be provided with a cannula which is designed for direct connection to the circulation of the subject. This advantageously facilitates the blood treatment directly in connection with the subject's body. Alternatively or in addition, the exchange device can be provided with a pump, which is arranged for transporting blood into or out of the container. This advantageously supports the movement of the blood sample.

According to a further preferred embodiment of the invention, the treatment apparatus is provided with a retaining device which is arranged for retaining the exogenous biological cell material in the container. The retaining device affords the advantage of excluding parts of the biological cell material, which were possibly inadvertently conveyed into the interior by the cell exposure device, being transported into the subject. Particularly preferably, the retaining device has a mechanically-acting filter (filter material which is exclusively impermeable to cells of the cell material) or a chemically-acting filter (surface to which exclusively the cell material binds specifically). Advantageously, the retaining device can be part of the exchange device, e.g. can be arranged on an outlet side of the exchange device.

According to a further advantageous embodiment of the invention, the treatment apparatus has a convection device which is designed for moving the blood sample in the interior of the container. Preferably, the blood sample is moved during treatment in the treatment apparatus. The convection device affords the advantage of increasing the effectiveness of the material interaction of the blood sample with the biological cell material by moving the blood sample. The convection device can e.g. comprise a stirring mechanism in the interior and/or a tilting mechanism, to which the container is coupled. If the container has at least one flexible container wall, the convection device can alternatively or in addition comprise a deformation device by means of which the container is deformed from the outside.

According to a further embodiment of the invention, if the treatment apparatus comprises a plurality of containers each having an exchange device and a cell exposure device which has biological cell material, the effectiveness of the immunotolerance enhancement can advantageously be increased. Particularly preferably, the containers contain different types of biological material, such that a broad enhancement of immunotolerance, i.e. a simultaneous enhancement of immunotolerance in relation to different antigens, is achieved.

Further advantages of the invention may arise if the treatment apparatus contains active substances which promote the development of immunotolerance, in particular by promoting regulatory T cells or acting on the mTOR system, and/or contain at least one anticoagulant substance and/or active substances which have an immunotolerance-enhancing action and/or have a supporting function in inducing immunotolerance. For example, active substances can be provided which strengthen a response of endogenous immune cells in the blood, prolong the residence time of specific blood components, enrich predetermined blood components, extend immunotolerance, inhibit blood coagulation, intensify immunotolerance and/or provide immunotolerance after returning the modulated blood (downstream strengthening).

According to a further preferred variant of the invention, the treatment apparatus can be provided with a measuring device which is designed to detect substances from the blood which are bound to the biological cell material, in particular antibodies. Advantageously, the treatment apparatus can be used for analytical or diagnostic purposes. For example, the amount of IgG antibodies which are bound to the exogenous or endogenous biological cell material can be measured. Here, in contrast to customary immunological assays (e.g. immunochromatographic assays, ELISA), selectively chosen antibody binding sites are not presented, but rather a plurality of possible binding sites. This enables wide-ranging analyses or early detection of an autoimmune disease.

Alternatively or in addition, the treatment apparatus can be provided with a collecting device which is designed to collect substances from the blood sample which are bound to the biological cell material, in particular antibodies. Advantageously, this makes it possible to use the treatment apparatus to carry out specific selection processes in the recipient blood. Here, either the components bound to exogenous cell material or non-binding components contained in the blood are collected and further processed.

Advantageously, there are different variants to provide the blood to be treated in the treatment apparatus. First, the blood sample to be treated, in particular based on the donation of the subject's own blood, is provided in a supply reservoir, in particular in a bag, and/or discharged into a collection reservoir, in particular into a bag. Advantageously, this enables the blood treatment to be carried out without the subject being present. For example, the recipient can donate blood multiple times even weeks or months before a planned transplantation, from which blood the blood samples are obtained for the treatment according to the invention. The treatment with the treatment apparatus takes place in each case in a laboratory, a clinic/treatment center, until a desired enhancement of the immunotolerance is achieved. In order to give immunotolerance before the transplantation, the treated blood is returned back to the recipient.

Second, the treatment apparatus can be connected to the subject's circulation, with the blood to be treated flowing into the treatment apparatus and back out therefrom into the circulation. Connection to the subject's circulation can comprise connection of at least one line of the exchange device via a cannula of the respective line directly to a blood vessel and/or a connection of at least one line of the exchange device to a fluid connector present beforehand at the blood vessel, e.g. cannula, catheter or port.

Further details and advantages of the invention will be described below with reference to the appended drawings. The drawings show:

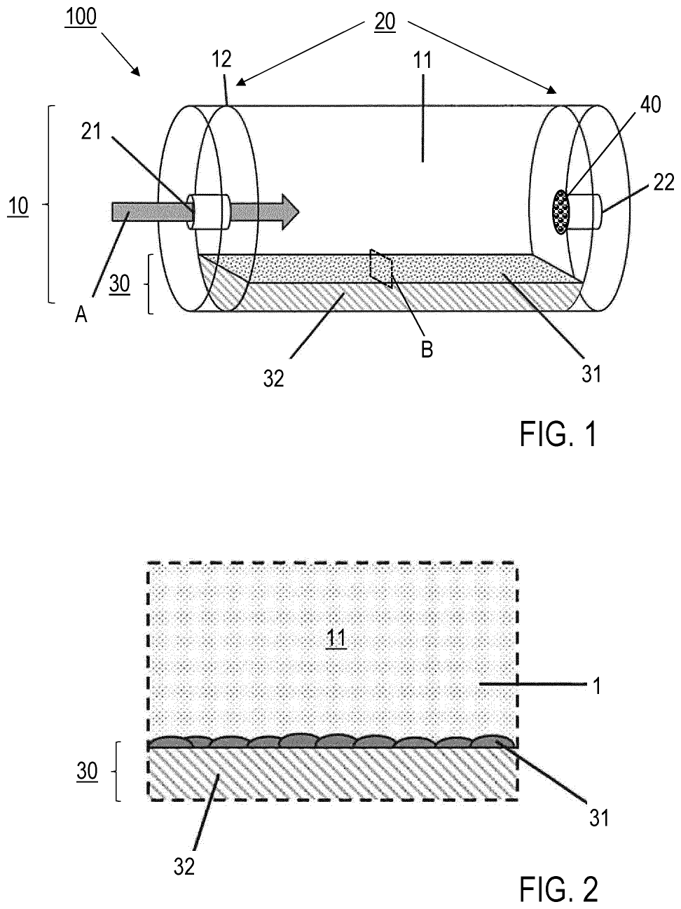

FIG. 1 a schematic overview of an embodiment of the treatment apparatus according to the invention;

FIGS. 2 to 4 features of further embodiments of the treatment apparatus according to the invention;

FIGS. 5 and 6 features of embodiments of the method according to the invention for extracorporeal blood treatment; and

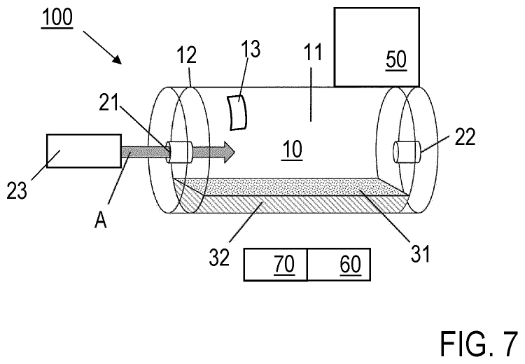

FIG. 7 a schematic overview of features of further embodiments of the treatment apparatus according to the invention.

Embodiments of the invention are described below with reference to the structure of the treatment apparatus and the application thereof. Details of the preparation of the cell material, e.g. the production and optional differentiation of iPS cells or adult stems cells are not described, since they are known per se. By way of example, reference is made to a treatment apparatus having a cylindrical container and a cell exposure device adjacent to the interior on one side. The implementation of the invention is not restricted to this embodiment, but rather is possible correspondingly with other container shapes and differently configured cell exposure devices.

FIG. 1 schematically shows an embodiment of the treatment apparatus 100, comprising a cylindrical container 10, which has an interior 11 for receiving a blood sample 1 and an exchange device 20, a cell exposure device 30 with biological cell material 31 and a retaining device 40. The end faces of the interior 11 are closed with sterile sealing walls 12. The exchange device 20 comprises an inlet line 21 and an outlet line 22 (only a portion of each of which is shown, see also FIGS. 5 and 6), which are arranged on the opposite end faces of the interior 11 and lead through the sealing walls 12. The inlet line and the outlet line each contain a closable valve (not shown) for separating the interior 11 from the surroundings, e.g. during the treatment of the blood sample 1. The interior 11 has a volume of for example 100 ml.

The cell exposure device 30 comprises the biological cell material 31 which is arranged adherently bound to a solid support 32. The biological cell material 31 comprises, e.g. in the treatment of the blood sample 1 before a kidney transplantation, differentiated iPS cells which are produced from donor tissue and differentiated to give kidney tissue. In the example illustrated, the biological cell material 31 has a free surface such that the blood sample 1 in the interior 11 directly touches the biological cell material 31 (see enlargement B from FIG. 1, in FIG. 2).

The retaining device 40 comprises a filter which is attached to the outlet line 22 and retains chemically and/or mechanically unintentionally released parts of the cell material 31 in the treatment apparatus 100.

The container 10 forms a volume which is filled via at least one inlet, such as the inlet line 11, with the blood sample 1 from the subject (see arrow A). As illustrated, the container 10 can be shaped cylindrically, or else in some other shape, e.g. cuboid or prism-shaped. After the incubation of the blood sample 1 in the interior 11 and in the presence of the exogenous cell material, e.g. for a duration of 4 hours, the blood sample 1 is removed either via the abovementioned inlet or a second inlet, such as the outlet line 22. Alternatively to the depiction in FIG. 1, it is possible to directly connect the treatment apparatus 100 to a cannula and to transfer the blood directly via the cannula into the container and back again into the subject. This advantageously reduces the risk of contamination.

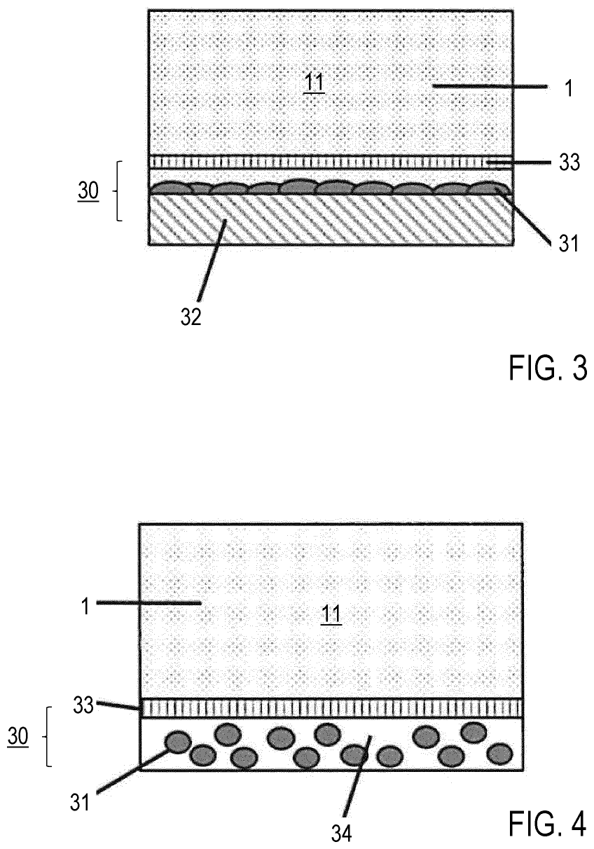

The biological cell material 31 is located in the container 10 on the support material 32, which cell material preferably has binding possibilities and presents these to the immune system components (e.g. T and B cells) in the blood sample 1. The exogenous cell material 31 can be mechanically stabilized (see FIG. 3) by a separating layer 33 lying over same, e.g. a membrane or frame structure. The support material 32 comprises polymers, biopolymers and/or hydrogels. Furthermore, anticoagulants can be introduced into the support material 32 and/or the interior 11, in order to reduce the risk of blood coagulation. Furthermore, active substances can be arranged in the support material 32 and/or in the interior 11, which promote the development of immunotolerance, e.g. the active substance rapamycin (see [2]).

Because the treatment apparatus 100 is intended to be used on site in medical institutions (doctor's surgeries, hospitals, laboratories), the container 10 as a whole can be frozen (e.g. below −130° C.) and stored. When the treatment apparatus 100 is then intended to be used, the thawing can take place for a specific period of time, or else the cells are directly thawed with the warm blood sample 1 from the subject. Alternatively, exclusively the support material 32 with the exogenous cell material 31 can be separately stored in a frozen state. In this case, the support material 32 with the exogenous cell material 31 is thawed shortly before use and transferred into the container 10, or is thawed directly in the container 10.

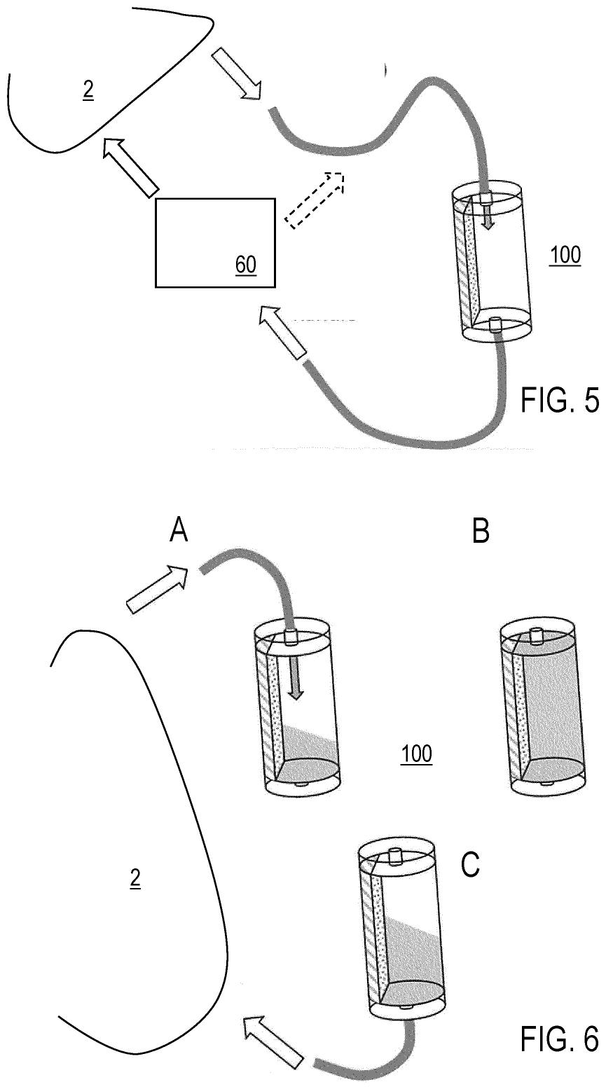

FIGS. 3 and 4 show alternative configurations of the cell exposure device 30 (corresponding to the section B from FIG. 1). In these embodiments, a separating layer 33 is provided, which separates the biological cell material 31 from the interior 11. Here, the biological cell material 31 can be securely attached to a support 32 (see FIG. 3) or arranged suspended in a suspension space 34 without support (see FIG. 4). The separating layer 33 is impermeable to the cells of the cell material 31 and the cell components thereof, such as cell membrane fragments, but permeable to antigen molecules. The separating layer 33 comprises e.g. polymers and/or hydrogels.

FIG. 5 illustrates a first method variant in which the treatment apparatus 100 is coupled more continuously with the circulation of the subject 2. Blood flows under the action of blood pressure, optionally aided by gravity and/or a pump (see FIG. 7), directly from the subject into the treatment apparatus 100 and from said treatment apparatus back to the subject 2. Here, a measuring device 60 can be provided in the outlet line, by means of which the immunotolerance achieved in the flowing blood is quantitatively detected. For example, antibodies (against major histocompatibility complexes) acting against the exogenous cell material can be measured in the subject's blood. For random sampling, e.g. an ELISpot measurement can be used. Depending on the measurement result, the blood can be returned to the treatment apparatus 100 again (see dashed arrow) in order to further enhance the immunotolerance.

Alternatively or in addition, the treatment apparatus 100, in particular the biological cell material therein, can also be used to evaluate the immune response. In this case, the adherent cells in the treatment apparatus 100 are analyzed after the blood reaction. To this end, the reduced viability of the adherent cells can be measured in an immune response. Furthermore, antibodies bound to the cells can be detected and quantified (e.g. by human antibodies directed against animal antibodies).

FIG. 6 illustrates a second method variant in which, in a first step (see FIG. 6A), the treatment apparatus 100 is loaded with a blood sample. The blood sample is, as in FIG. 5, taken directly from the subject or added to a supply reservoir (not shown) by a blood donation and transferred from the supply reservoir to the treatment apparatus 100. Subsequently, the blood sample is incubated (without connection to the subject 2) in the treatment apparatus 100 (see FIG. 6B). After the treatment of the blood sample, the blood is returned to the subject 2 (see FIG. 6C). In this case, too, a measurement to evaluate the immune response can be provided on the blood and/or cell material.

FIG. 7 shows further components of the treatment apparatus 100, which can be provided individually or in combination. The treatment apparatus 100 is as shown in FIG. 1. In addition, a pump 23 is provided, by means of which the blood flow through the container 10 is aided. Furthermore, a schematically depicted closable access opening 13, for example for measuring and/or monitoring purposes, can be provided in a container wall.

The schematically depicted convection device 50 comprises e.g. a tilting and/or deforming mechanism, by means of which the container 10 can be moved and/or deformed in order to move the blood sample 1 in the interior 11 of the container 10. Alternatively or in addition, a magnetic stirrer (not shown) can be provided in the interior.

FIG. 7 further schematically depicts the measuring device 60 which is designed to detect substances from the blood sample 1 which are bound to the biological cell material 31, in particular antibodies (see FIG. 5) and a collecting device 70 which is designed to collect substances from the blood sample 1 which are bound to the biological cell material 31, in particular antibodies, or non-binding components present in the blood.

The features of the invention disclosed in the above description, the drawings and the claims, whether individually or in combination or sub-combination, can be relevant to the implementation of the invention in its various configurations.

1. A treatment apparatus, which is designed for extracorporeal blood treatment of blood from a subject for an immunotolerance enhancement of the subject, comprising:
at least one container which has an interior for receiving a blood sample of blood from the subject and an exchange device which is designed to supply and/or discharge the blood sample into or out of an interior of the at least one container, and
a cell exposure device which is positioned in the at least one container and has biological cell material with surface molecules, wherein the biological cell material is arranged in the interior of the at least one container for a material interaction with the blood sample, comprising a reaction of endogenous immune cells in the blood with the surface molecules of the biological cell material.

2. The treatment apparatus according to claim 1, wherein
the cell exposure device has a separating layer, wherein the biological cell material is separated from the interior by the separating layer and the biological cell material is held on the cell exposure device and the separating layer is designed for a substance exchange between the biological cell material and the blood sample.

3. The treatment apparatus according to claim 1, wherein
the cell exposure device has a support on which the biological cell material is arranged in an adherent state.

4. The treatment apparatus according to claim 3, wherein
the support has a binding layer which is designed for an adherent binding of at least one of induced pluripotent stem cells (iPS cells), adult stem cells and cells differentiated therefrom.

5. The treatment apparatus according to claim 1, wherein
the cell exposure device has a suspension space in which the biological cell material is arranged in at least one of a suspended state and/or on carrier beads.

6. The treatment apparatus according to claim 1, wherein the biological cell material comprises at least one of:
exogenous biological cell material,
endogenous biological cell material from the subject,
at least one of cell-equivalent iPS cells and iPS cell constituents,
at least one of organ-equivalent differentiated iPS cells and iPS cell components,
organ-equivalent differentiated adult stem cells,
at least one of biological cells and/o cell components which trigger immune responses in the blood,
at least one of exogenous cells which are provided for allogeneic transplant into the subject, and components thereof,
a composition of cell material which contains different cell types which carry characteristic HL antigens from a predetermined reference group of subjects, and
cells which have been genetically modified to undergo cell death which can be stimulated at least one of externally and in a time-dependent manner.

7. The treatment apparatus according to claim 1, wherein the exchange device comprises at least one of the features:
the exchange device has an inlet line and an outlet line which are in fluidic connection with the interior end are arranged mutually spaced apart,
the exchange device is provided with a cannula which is designed for direct connection to the circulation of the subject, and
the exchange device is provided with a pump which is arranged for blood transport into or out of the container.

8. The treatment apparatus according to claim 1, which comprises
a retaining device which is arranged for retaining the biological cell material in the container.

9. The treatment apparatus according to claim 8, wherein
the retaining device has a mechanically- or chemically-acting filter.

10. The treatment apparatus according to claim 8, wherein
the retaining device is part of the exchange device.

11. The treatment apparatus according to claim 1, which comprises
a convection device which is designed for moving the blood sample in the interior of the at least one container.

12. The treatment apparatus according to claim 11, wherein
the convection device comprises at least one of a stirring mechanism in the interior and a tilting mechanism, to which the at least one container is coupled.

13. The treatment apparatus according to claim 11, wherein
the at least one container has a flexible container wall and the convection device comprises a deformation device by way of which the at least one container is deformed.

14. The treatment apparatus according to claim 1, comprising
a plurality of containers each with an exchange device and a cell exposure device, which has biological cell material, wherein
the containers contain different types of biological material.

15. The treatment apparatus according to claim 1, which contains at least one of
active substances which promote the development of immunotolerance,
active substances which promote regulatory T cells,
active substances which act on the mTOR system,
at least one anticoagulant substance, and
active substances which have at least one of an immunotolerance-enhancing action and a supporting function in inducing immunotolerance.

16. The treatment apparatus according to claim 1, which comprises at least one of
a measuring device which is designed to detect substances from the blood sample which are bound to the biological cell material, and
a collecting device which is designed to collect substances from the blood sample which are bound to the biological cell material.

17. A kit for extracorporeal blood treatment, comprising at least one treatment apparatus according to claim 1.

18. A method for operating a treatment apparatus according to claim 1 for extracorporeal blood treatment of blood from a subject for an immunotolerance enhancement of the subject, comprising the steps of:
providing a blood sample to be treated of blood from the subject,
introducing the blood sample into the at least one container of the treatment apparatus,
treating the blood sample by an interaction, comprising a reaction of endogenous immune cells with the surface molecules of the presented biological cell material such that the blood sample after treatment is suitable for effecting an enhanced immunotolerance of the subject, and
discharging the blood sample from the container.

19. The method according to claim 18, wherein
the blood sample to be treated is at least one of provided in a supply reservoir and discharged into a collection reservoir.

20. The method according to claim 18, wherein
the treatment apparatus is connected to the circulation of the subject, wherein
the blood to be treated flows as the blood sample into the treatment apparatus and back out therefrom into the circulation.

21. The method according to claim 18, wherein
the blood sample is moved during the treatment in the treatment apparatus.

22. The method according to claim 18, comprising the step of
adjusting at least one of an amount and a concentration of the biological cell material in the treatment apparatus depending on a given or achieved immunotolerance of the subject.

23. The method according to claim 18, wherein
the treatment apparatus is provided in a frozen state and is thawed before the blood sample is introduced into the container of the treatment apparatus.

24. The method according to claim 18, wherein
a blood donation from the subject is at least one of provided in a supply reservoir and discharged into a collection reservoir.

25. The method according to claim 18, wherein
the cell exposure device with the biological cell material is provided in a frozen state and is thawed before the blood sample is introduced into the at least one container of the treatment apparatus.
